(19) United States
(12) Patent Application Publication
(10) Pub. No.: US 2005/0201910 A1
(43) Pub. Date: Sep. 15, 2005

(54) Mobile disinfection apparatus

(76) Inventors: Ming - Hwa Shou, Sindian City (TW); Ming - Song Chou, Sindian City (TW)

Correspondence Address: Yi-Wen Tseng, 4331 Stevens Battle Lane, Fairfax, VA, 22033, US

(21) Appl. No.: 10/798,507

(22) Filed: Mar. 12, 2004

(51) Int. Cl. A61L 2/00; A62B 7/08; B01J 19/08; A61L 9/00

(52) U.S. Cl. 422/292; 422/24; 422/28; 422/121; 422/186; 422/300; 422/306

(57) ABSTRACT

A mobile disinfection apparatus is provided to disinfect the sickbeds. The disinfection apparatus includes a retractable hollow shield with a plurality of wheels for enclosing the sickbed thereinside; a disinfector furnished on an inner surface of the shield for disinfecting the sickbed; and a control unit with a control panel located outside the shield for activating the disinfector.

### BACKGROUND OF THE INVENTION

The present invention relates in general to a mobile disinfection apparatus, and more particularly, to a disinfection apparatus to be used in disinfecting the sickbeds, which is easy in traveling and for storage, and can rapidly complete the disinfection work with minimum manpower.

The hospital and nursing home provide beds for sick persons. The sickbeds are public equipments so that it is very important to make sure the bed is thoroughly disinfected after it is used by others. Therefore, it exists demand of a special disinfection apparatus to disinfect the sickbeds in convenience.

### BRIEF SUMMARY OF THE INVENTION

The present invention provides a mobile disinfection apparatus for a sickbed, which is easy in traveling and for storage, and can rapidly complete the disinfection work with minimum manpower.

Further, the present invention provides a disinfection apparatus for disinfecting the sickbed from the under side, which can be cooperated with the above-mentioned mobile disinfection apparatus to make sure the sickbed is thoroughly disinfected thereby.

Accordingly, the mobile disinfection apparatus for a sickbed includes a retractable hollow shield with a plurality of wheels for enclosing the sickbed thereinside; a disinfector furnished on an inner surface of the shield for disinfecting the sickbed; and a control unit with a control panel located outside the shield for activating the disinfector.

Furthermore, the disinfection apparatus for disinfecting a under portion of a sickbed includes a base located under the sickbed; a disinfector furnished on a top surface of the base for disinfecting the under portion of the sickbed; and a control unit with a controller for activating the disinfector.

These and other objectives of the present invention will become obvious to those of ordinary skill in the art after reading the following detailed description of preferred embodiments.

It is to be understood that both the foregoing general description and the following detailed description are exemplary, and are intended to provide further explanation of the invention as claimed.

### BRIEF DESCRIPTION OF THE DRAWINGS

These, as well as other features of the present invention, will become apparent upon reference to the drawings wherein:

FIG. 1 shows a perspective view of a mobile disinfection apparatus in an unfolding status according to the present invention;

FIG. 2 shows a different view of the mobile disinfection apparatus in FIG. 1;

FIG. 3 shows an enlarged view of part A of the mobile disinfection apparatus in FIG. 2;

FIG. 4 shows a perspective view of another cooperated disinfection apparatus according to the present invention;

FIG. 5 shows an application of the disinfection apparatus for a sickbed;

FIG. 6 shows a front view of the disinfection apparatus in FIG. 5; and

FIG. 7 shows a perspective view of the mobile disinfection apparatus in a folding status.

### DETAILED DESCRIPTION OF THE INVENTION

Reference will now be made in detail to the preferred embodiments of the present invention, examples of which are illustrated in the accompanying drawings. Wherever possible, the same reference numbers are used in the drawings and the description to refer to the same or like parts.

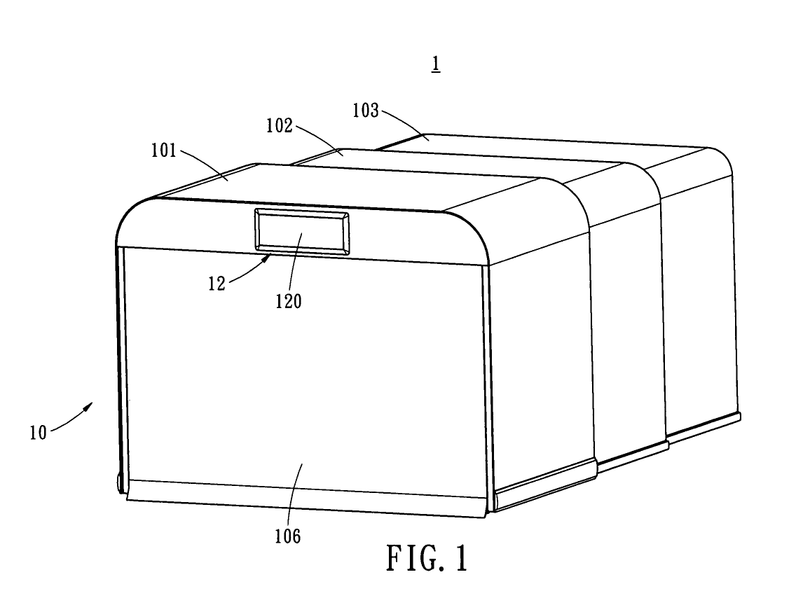

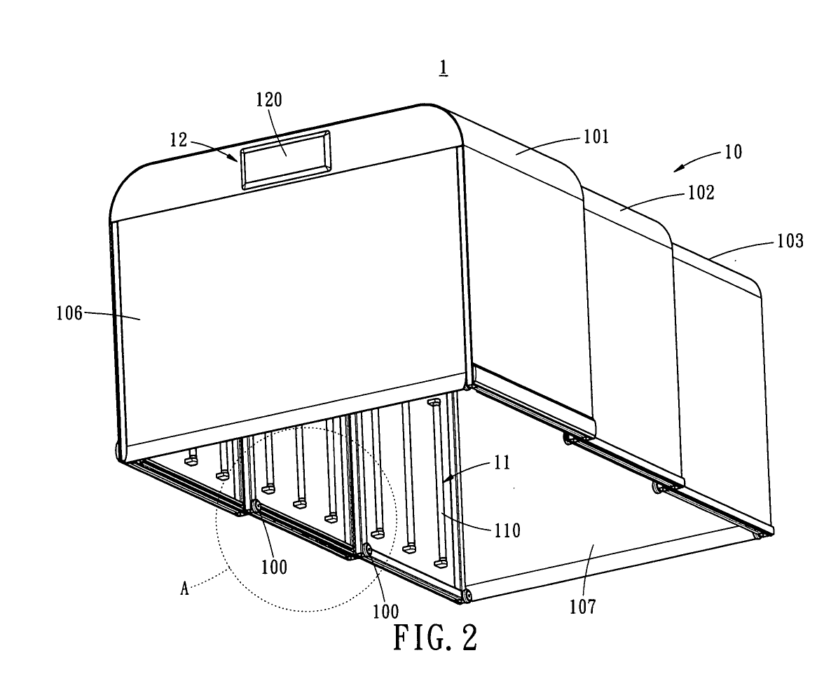

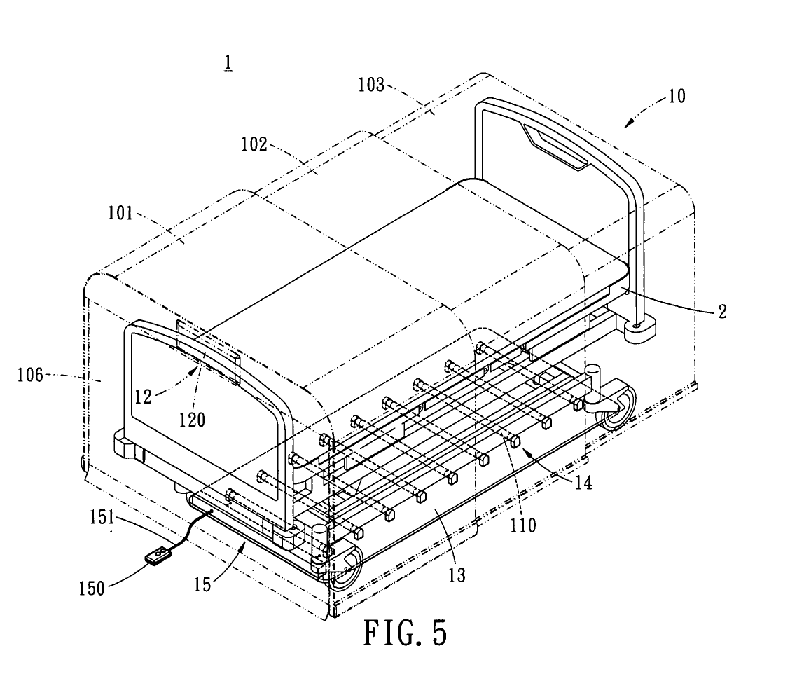

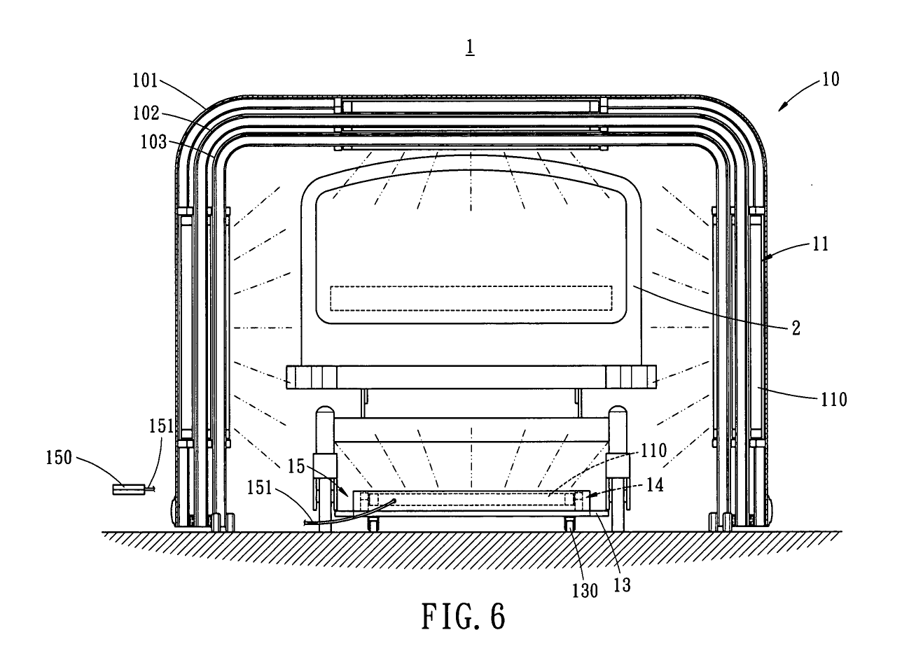

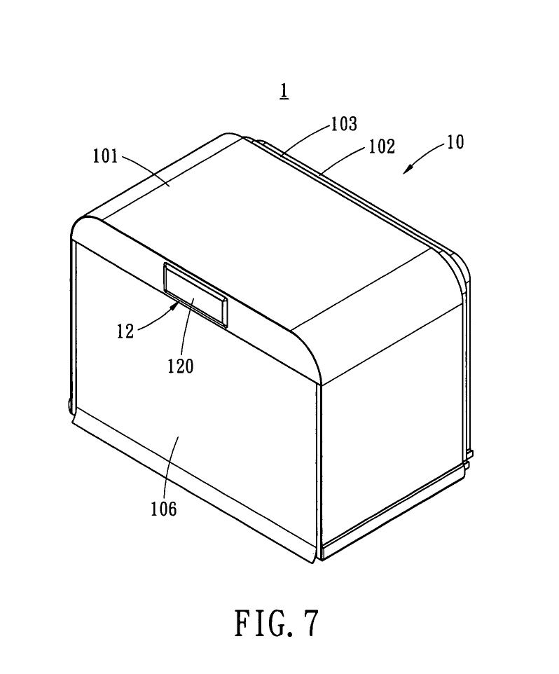

Please refer to FIGS. 1 and 7, which show the perspective views of an unfolding and a folding disinfection apparatus of the present invention, respectively. The mobile disinfection apparatus 1 of the present invention includes a retractable hollow shield 10 for enclosing a sickbed 2 (as shown in FIG. 5 or 6) inside the shield 10. Therefore, the sickbed 2 can be entirely shielded to be disinfected under the enclosure of the retractable shield 10. The shield 10 is foldable to be expanded for use as in FIG. 1, or to be folded for storage as in FIG. 7. Moreover, as shown in FIG. 2, the shield 10 has a plurality of wheels 100 to provide the mobility of the disinfection apparatus 1 in traveling to another sickbed.

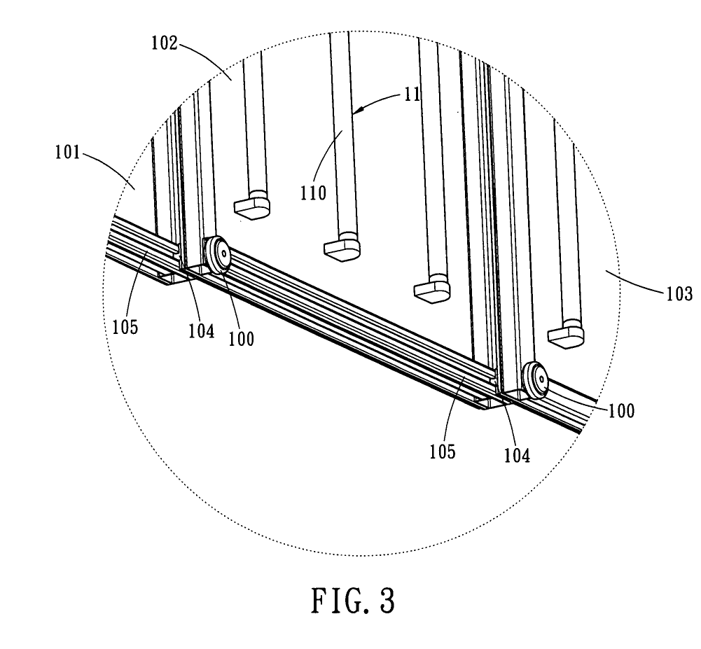

As shown in FIGS. 1 and 2 of an preferred embodiment of the present invention, the retractable shield 10 includes a plurality of n-shaped casings 101, 102, 103 connected to each other. Referring to FIG. 3, each two casings 101, 102 or 102, 103 are connected by an engagement of a rail 105 of one casing 102 or 103 to a sliding slot 104 of the other casing 101 or 102, respectively. As such, the retractable shield 10 can be easily unfolded for use and folded for storage. Furthermore, at both front end 106 and rear end 107 of the retractable shield 10 can be constructed to be opened upwards or aside (either from right side or left side). Therefore, the sickbed 2 can be put inside the shield 10 from opening the front side 106 of the rear side 107. However, of course there can be only one side of the shield capably to be opened and the other side is closed.

As in FIGS. 2, 3 and 6, the mobile disinfection apparatus 1 includes a disinfector 11 to kill bacteria and virus. The disinfector 11 includes at least one of a high-temperature vapor, a light exposure, a medication, a physical/chemical disinfection device or any other disinfector capably to kill and/or inactivate germs. In this preferred embodiment, the disinfector 11 includes a plurality of ultra violet (UV) light tubes 110 which provide UV light to kill and/or inactivate germs. The UV tubes 110 are mounted on each inner wall of the casings 101, 102, 103. The inner wall of each casing 101, 102 or 103 is recessed, as shown in FIG. 3, for the UV tubes 110 to be installed therein. As such, when the mobile disinfection apparatus is folded, the casings 101, 102, 103 will not interfere to each other for the retraction of the shield 10. In this embodiment, the UV tubes 110 are preferably the UV254 tubes which provide an ultra violet light of 253.7 nm wavelength.

Back to FIGS. 1 and 2, the disinfector 11 is controlled by a control unit 12 to switch on/off, and determine the power and/or the operation time of the disinfector 11. The control unit 12 includes a control panel mounted outside the shield 10 for the operation of the users.

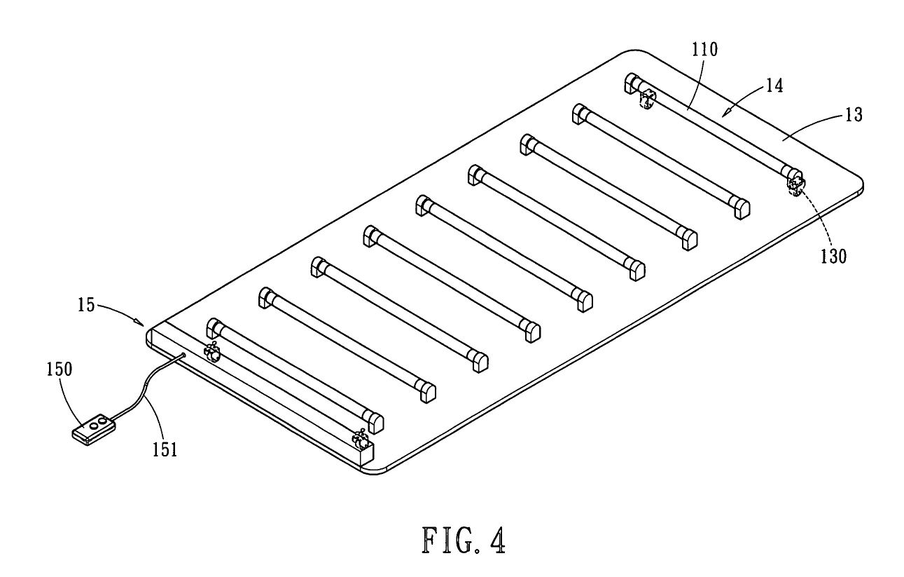

Furthermore, as in FIGS. 4 and 6, the disinfection apparatus of the present invention can further includes a plate base 13 and another disinfector 14 mounted on the base 13. This disinfector 14 is similar to the above-mentioned disinfector 11, which includes at least one of a high-temperature vapor, a light exposure, a medication, a physical/chemical disinfection device or any other disinfector capably to kill and/or inactivate germs. In this embodiment, the disinfector 14 includes a plurality of ultra violet (UV) light tubes 110. The base 13 also includes a plurality of wheels 130 so that the base 13 can be movably located under the sickbed 2, and the disinfector 14 is provided to disinfect a under portion of the sickbed 2. Moreover, another control unit 15 is also provided to switch on/off, and determine the power and/or the operation time of the disinfector 14. The control unit 15 includes a controller 150 which can further includes a cord 151 to connected with the control unit 15 or connect to the control unit 15 via wireless connection.

Finally, please refer to FIGS. 5 and 6, an application of the present invention are shown. Using both the shield 10 and the base 13, the sickbed 2 can be thoroughly disinfected in convenience. The shield 10 is used to disinfect both the right and left sides of the sickbed 2, and the base 13 is used to disinfect the sickbed 2 from the under side. As such, the disinfection work can be rapidly complete in minimum time without manually wiping and cleaning action. After the disinfection, the sickbed 2 is moved out from the mobile disinfection apparatus of the present invention, and a clean mattress, sheet and pillow can be then placed on the sickbed for the next use.

This disclosure provides exemplary embodiments of the present invention. The scope of this disclosure is not limited by these exemplary embodiments. Numerous variations, whether explicitly provided for by the specification or implied by the specification, such as variations in shape, structure, dimension, type of material or manufacturing process may be implemented by one of skill in the art in view of this disclosure.

1. A mobile disinfection apparatus for a sickbed, comprising:
a retractable hollow shield with a plurality of wheels for enclosing the sickbed thereinside;
a disinfector furnished on an inner surface of the shield for disinfecting the sickbed; and
a control unit with a control panel located outside the shield for activating the disinfector.

2. The apparatus of claim 1, wherein the shield includes a plurality of casings connected to each other.

3. The apparatus of claim 2, wherein each two casings are connected by an engagement of a rail of one casing to a sliding slot of the other.

4. The apparatus of claim 1, wherein the shield includes two ends, and at least one end is openable.

5. The apparatus of claim 4, wherein the other end of the shield is closed.

6. The apparatus of claim 1, wherein the disinfector includes at least one of a high-temperature vapor, a light exposure, a medication, or a physical/chemical disinfection device.

7. The apparatus of claim 1, wherein the disinfector includes a plurality of ultra violet light tubes.

8. The apparatus of claim 7, wherein the shield includes a plurality of casings connected to each other, and each casing has a recessed inner wall for the ultra violet light tubes to be installed thereon.

9. The apparatus of claim 7, wherein the ultra violet light tube is a UV254 tube which provides an ultra violet light of 253.7 nm wavelength.

10. The apparatus of claim 1, wherein the control unit is further used to control a power or an operation time of the disinfector.

11. A disinfection apparatus for disinfecting a under portion of a sickbed, comprising:
a base located under the sickbed;
a disinfector furnished on a top surface of the base for disinfecting the sickbed; and
a control unit with a controller for activating the disinfector.

12. The apparatus of claim 11, wherein the base is a plate.

13. The apparatus of claim 11, wherein the base includes a plurality of wheels.

14. The apparatus of claim 11, wherein the disinfector includes at least one of a high-temperature vapor, a light exposure, a medication, or a physical/chemical disinfection device.

15. The apparatus of claim 11, wherein the disinfector includes a plurality of ultra violet light tubes.

16. The apparatus of claim 15, wherein the ultra violet light tube is a UV254 tube which provides an ultra violet light of 253.7 nm wavelength.

17. The apparatus of claim 11, wherein the control unit is further used to control an operation power or an operation time of the disinfector.

18. The apparatus of claim 11, wherein the controller is connected to the control unit via a cord.

19. The apparatus of claim 11, wherein the controller is connected to the control unit via a wireless connection.
